(19) United States
(12) Patent Application Publication
(10) Pub. No.: US 2021/0008244 A1
(43) Pub. Date: Jan. 14, 2021

(54) Adhesive Composition

(71) Applicant: RIJKSUNIVERSITEIT GRONINGEN, Groningen (NL)

(72) Inventors: Maria Magdalena Gerdina KAMPERMAN, Aduard (NL); Marco DOMPÈ, Manchester (GB); Alexei Dmitrievitsj FILIPPOV, Wageningen (NL)

(73) Assignee: Rijksuniversiteit Groningen, Groningen (NL)

(21) Appl. No.: 16/971,863

(22) PCT Filed: Mar. 8, 2019
(86) PCT No.: PCT/NL2019/050150

§ 371 Date: Aug. 21, 2020

(30) Foreign Application Priority Data
Mar. 9, 2018 (EP) 18161093.2

(51) Int. Cl. A61L 24/00; A61L 24/04

(52) U.S. Cl. CPC A61L 24/0031; A61L 24/0015; A61L 24/043

(57) ABSTRACT

The invention is directed to an adhesive complex coacervate composition, to a method of physically crosslinking an adhesive complex coacervate composition, to a method for adhering a tissue defect in a subject, and to the use of an adhesive complex coacervate composition. The adhesive complex coacervate composition of the invention comprises a polycation and a polyanion, wherein said polycation and polyanion together comprise on average at least two thermoresponsive moieties per polymer chain, said thermoresponsive moieties exhibiting a lower critical solution temperature, wherein said polycation comprises 5-70 mol % of thermoresponsive moieties and/or wherein said polyanion comprises 5-70 mol % of thermoresponsive moieties, and wherein said polycation and/or said polyanion is a graft or block copolymer comprising said thermoresponsive moieties.

The invention is directed to an adhesive complex coacervate composition, to a method of solidifying an adhesive complex coacervate composition, to a method for adhering a tissue defect in a subject, and to the use of an adhesive complex coacervate composition.

The last century has seen a tremendous advancement in adhesive technology and has led to extensive replacement of mechanical fasteners with adhesive bonds (e.g. aircraft, automobile, construction, etc.). Adhesive technology is used in a wide variety of applications. In (orthopaedic) medicine, however, adhesive technology is rarely applied. Conventional suturing and tissue stapling remain the standard method of surgical tissue closure and sealing, despite technical limitations. More specifically, sutures are difficult to place in small spaces, extend operating times and do not work effectively for many types of tissue (e.g. lung tissue). Staples can cause significant tissue damage and scarring. Surgical adhesives exist, but are primarily used for stopping bleeding and for gluing together skin externally. Deep tissue bonding remains challenging because of the wet and dynamic environment inside the body, and high-performance adhesives have not met the demands of (1) strong adhesion and cohesion; (2) controlled and precise delivery; and (3) biocompatibility. Current clinically available surgical glues include fibrin sealants, polycyanoacrylates, poly(ethylene glycol) based hydrogels and protein-glutaraldehyde glues. The available formulations have significant limitations: they exhibit poor adhesive properties, are toxic or can be easily washed out under in vivo conditions. Major efforts are ongoing to create new concepts, which recently resulting in promising developments such as TissuGlu (a polyurethane slowly curing by moisture ingress (www.coherameclical.com/productshissuglu) and Gecko Biomedical GB02 (a UV-curable poly(glycerol sebacate acrylate)) (Lang et al., Science Translational Medicine 2014, 6(218), 218ra6). The development of practical and efficient adhesives has the potential to greatly enhance surgical competency and reduce the incidence of complications. Adhesives that can be used and are effective in wet conditions are also useful in many non-medical applications.

In the aquatic world, several organisms have developed protein-based adhesives able to overcome similar challenges as faced by adhesive developers. A key element in the adhesive processing is polyelectrolyte complex formation, so-called complex coacervation. Complex coacervates are concentrated polymer phases of positively and negatively charged polymers (polyelectrolytes). As soon a complex is formed the charges cancel each other and a somewhat hydrophobic polymer complex remains This results in phase separation in which a water insoluble complex coacervate is formed together with a dilute phase (containing mainly water).

Additional covalent or strong non-covalent interactions may be required to achieve the required cohesive properties and turn the material into a tough and strong material. These additional interactions should be activated after delivery of the adhesive, i.e. an in-situ setting system. In the natural marine organisms, the setting is, for instance, triggered by a higher pH in seawater, or exposure to oxygen.

Synthetic complex coacervates are, e.g., known from WO-A-2016/011028. This document discloses high ionic strength adhesive complex coacervates that convert to an adhesive solid or gel when complex coacervate is introduced into a lower ionic strength application site.

A method for separating solid content from a suspension is known from JP-A-2012 170 871. In accordance with this method to the suspension is added a mixture of cationic temperature sensitive polymer and anionic temperature sensitive polymer. The polymers change from a hydrophilic nature to a hydrophobic nature when the temperature exceeds a transition temperature, thereby agglomerating solid material in the suspension which can then be separated. The cationic and anionic polymers are random copolymers wherein thermoresponsive monomers are randomly distributed in the copolymer.

Objective of the invention is to address this above expressed desire of providing an engineering material with a similar adhesion mechanism as some aquatic organisms.

Further objective of the invention is to provide an adhesive composition that is effective in an aqueous environment.

Yet a further objective of the invention is to provide an adhesive composition that can physically crosslink upon an environmental trigger.

Yet a further objective of the invention is to provide an adhesive composition that provides excellent adhesive strength in wet conditions.

The inventors found that one or more of these objectives can, at least partly, be met by providing an adhesive complex coacervate composition based on polyelectrolytes having thermoresponsive moieties.

Accordingly, in a first aspect the invention is directed to an adhesive complex coacervate composition comprising a polycation and a polyanion, wherein said polycation and polyanion together comprise on average at least two thermoresponsive moieties per polymer chain, said thermoresponsive moieties exhibiting a lower critical solution temperature, wherein said polycation comprises 5-70 mol % of thermoresponsive moieties and/or wherein said polyanion comprises 5-70 mol % of thermoresponsive moieties, and wherein said polycation and/or said polyanion is a graft copolymer or block copolymer comprising said thermoresponsive moieties.

The adhesive complex coacervate composition of the invention can solidify when triggered by heating. It is believed that complex coacervation contributes to the processing and performance of the adhesive, because (i) the immiscibility with water ensures that the adhesive remains at the application site during the curing process even when fully submerged in water, (ii) the interfacial tension of complex coacervates is very low, which enables the viscous fluid to readily wet the surface, (iii) water is readily displaced from the substrate to maximise adhesion, (iv) the dimensional stability when submerged; depending on conditions complex coacervates do not swell when submerged, and (v) charged additives can be easily mixed in and taken up by the formed complex coacervates.

The adhesive complex coacervate composition advantageously allows bonding in wet conditions and combines the great potential of complex coacervates with a self-assembling setting mechanism that is triggered by a change in temperature. Some advantages of the adhesive composition of the invention include that
(1) the adhesive may be delivered as a liquid through small-bore needles, while ensuring minimum washout, depending on the amount of salt and concentration,
(2) the adhesive can be solidified upon heating within a body without the need of external activation of the setting,
(3) the adhesive can strongly bind to various substrates, and
(4) the adhesive is able to withstand applied mechanical stress.

The bio-inspired synthetic adhesive comprises a blend of oppositely charged polyelectrolytes with responsive domains. The adhesive starts out as a fluid and self-assembles into a solid upon heating above the lower critical solution temperature of the thermoresponsive moieties, which causes the polymers to associate. Due to its complex coacervate nature, the adhesive displaces water from the substrate and spreads easily onto the substrate. In addition, the adhesive remains at the application site during solidification even when fully submerged in water. This can result in an adhesive that may be delivered via minimal invasive procedures with good performance within the dynamic environments inside the body.

Notably, curing is performed via an external trigger, viz. an increase in temperature. Accordingly, no additional components have to be added to the adhesive composition for curing. In other words, the invention advantageously relates to a one component system.

Besides the self-assembling setting mechanism and the intrinsic advantages of complex coacervates for underwater adhesion, this material system has further advantageous elements.

To enable delivery through small bore needles, the viscosity of the adhesive can be kept low by increasing the salt concentration (ionic strength). That is, the viscosity of complex coacervates is strongly dependent on ionic strength. At high ionic strength, the viscosity can be as low as 1 Pa·s (which resembles the viscosity of glycerol). Drastically decreasing the ionic strength will transition the liquids to stiff gels. Therefore, the viscosity of the adhesive composition of the invention can be controlled and optimised for a specific application by adjusting the ionic strength. Delivery into the lower physiological ionic strength will subsequently increase the viscosity.

Furthermore, polymer chains containing amphiphilic and ionic features are self-adjustable. This means that, depending on the target surface, different parts of the polymer will be exposed to the surface. This is believed to ensure excellent adhesive bonding to a variety of different tissues.

Unlike most adhesives that are under development, the final solid material is held together by non-covalent interactions. The different types of interactions will result in a wide variety of bond strengths. These types of materials can be extremely strong, tough, and self-healing, despite the lack of covalent bonds. The behaviour is completely different from a typical non-covalent hydrogel that is considered mechanically weak; the key difference being a single versus a variety of bond strengths.

Complex coacervates are further ideally suited to include charged bioactive agents. Incorporation of bioactive molecules such as drugs can enhance healing of the tissue. In conventional adhesives, composed of uncharged polymer networks, blending is difficult, due to polymer incompatibility. For polymers, just a very small unfavourable interaction per monomer is enough to induce unwanted phase separation between polymer and additive. A big advantage of the complex coacervate system of the invention is that polymer incompatibility is much less of a problem for charged polymers. Charged molecules may easily be mixed in, to be taken up by the formed complex coacervate, as long as the complete system contains approximately as many positive as negative charges.

The adhesive complex coacervate composition of the invention comprises a polycation and a polyanion, wherein the polycation and the polyanion together on average comprise at least two thermoresponsive moieties per polymer chain. This means that on average there are at least two thermoresponsive moieties per polymer chain The thermoresponsive moieties may be present on the polycation, on the polyanion, or both. It is preferred that the polycation comprises at least two thermoresponsive moieties and that the polyanion also comprises at least two thermoresponsive moieties. The term “polycation” as used herein is meant to refer to a positively charged polyelectrolyte, whereas the term “polyanion” is meant to refer to a negatively charged polyelectrolyte. The polycation and polyanion can both or individually be a copolymer comprising the thermoresponsive moieties. The polycation and/or the polyanion is a graft copolymer or a block copolymer. In such copolymers the thermoresponsive moieties form thermoresponsive domains. The polycation can be graft copolymer or block copolymer comprising thermoresponsive domains. Additionally, or alternatively, the polyanion can be a graft copolymer or block copolymer comprising thermoresponsive domains. In a graft copolymer, the thermoresponsive domains are present as side branches on the primary polymer chain. In a block copolymer the thermoresponsive domains are present as blocks in the primary polymer chain, typically at both ends thereof. This is contrary to a random copolymer, wherein the thermoresponsive moieties are present as monomeric units randomly distributed in the primary polymer chain. Graft copolymer and/or block copolymers allow the formation of domains rich in thermoresponsive moieties and domains poor in thermoresponsive moieties. Without wishing to be bound by any theory, it is believed that the domains rich in thermoresponsive moieties self-aggregate which in turn leads to the formation of physical crosslinks. The inventors found that such self-aggregation of thermoresponsive moieties is significantly better when domains are formed.

The polycation comprised in the adhesive complex coacervate composition of the invention can be composed of a polymer backbone with a plurality of cationic groups. The cationic groups can be pendant to the polymer backbone and/or incorporated within the polymer backbone. Optional thermoresponsive moieties may be introduced into the polycation by graft copolymerisation or block copolymerisation.

The polycation can, for example, be a polyamine compound. The amino groups of a polyamine can be branched or part of the polymer backbone. The amino groups may be primary, secondary, or tertiary amino groups that can be protonated to produce a cationic ammonium group at a selected pH. In general, a polyamine is a polymer with a large excess of positive charges relative to negative charges at the relevant pH, as reflected by its isoelectric point, which is the pH at which the polymer has a net neutral charge. The number of amino groups present on the polycation ultimately determines the charge of the polycation at a particular pH. For example, the polycation can have 10-90 mol %, 10-70 mol %, 10-50 mol %, 10-30 mol %, or 10-20 mol % of amino groups. The polyamine can for example have an excess positive charge at a pH of about 7, with an isoelectric point significantly greater than 7. Additional amino groups can be incorporated into the polymer in order to increase the pKₐ. The amino group can be derived from a residue of lysine, histidine, or arginine attached to the polycation.

In a special embodiment, the polycation is a biodegradable polyamine. The biodegradable polyamine can be a synthetic polymer, a genetically engineered polymer (i.e. polymers that are synthesised by recombinant DNA technology), a naturally-occurring polymer, or combinations thereof. The mechanism by which the polyamine can degrade will vary depending upon the polyamine that is used. In the case of natural polymers, they are typically biodegradable because there are enzymes that can hydrolyse the polymers and break the polymer chain. For example, proteases can hydrolyse natural proteins like gelatine. Synthetic biodegradable polyamines typically possess chemically labile bonds. For examples, (β-aminoesters have hydrolysable ester groups. In addition to the nature of the polyamine, other consideration such as the molecular weight of the polyamine and crosslink density of the adhesive can be varied in order to modify the degree of biodegradability.

The polycation, in particular a biodegradable polyamine, may comprise a polysaccharide, a protein, or a synthetic polyamine. Polysaccharides bearing one or more amino groups can be used. In one aspect, the polysaccharide is a natural polysaccharide such as chitosan or chemically modified chitosan. Similarly, the protein can be a synthetic or naturally-occurring compound. The biodegradable polyamine can, for instance, be a synthetic polyamine such as poly((β-aminoesters), polyester amines, poly(disulphide amines), mixed poly(ester and amide amines), and peptide crosslinked polyamines.

In case the polycation is a synthetic polymer, a variety of different polymers can be used. It is preferred, however, that the polycation is biocompatible, and non-toxic to cells and tissue. The biodegradable polyamine can be an amino-modified natural polymer. For example, the amine-modified natural polymer can be gelatine modified with one or more alkylamino groups, heteroaryl groups, or an aromatic group substituted with one or more amino groups.

The polycation can further comprise a polyacrylate having one or more pendant amino groups. For example, the backbone of the polycation can be derived from the polymerisation of acrylate monomers, such as acrylates, methacrylates, acrylamides, methacrylamides, vinyl esters, vinyl ethers, vinyl amides, and the like. The polycation backbone can be derived from polyacrylamide. The polycation can further be a block copolymer, where segments or portions of the copolymer possess cationic groups or neutral groups depending upon the selection of the monomers used to produce the copolymer.

The polycation can also be a polyamino compound. Such a polyamino compound can, for example, have 10-90 mol % of primary amino groups, such as 10-70 mol %, 10-50 mol %, or 10-30 mol %.

Suitable examples of specific polycations (excluding thermoresponsive moieties) include cationic polypeptides, poly(L-lysine) polycations, poly(D-lysine) polycations, cationic polysaccharides, diethylaminoethyl dextran polycations, cationic starch, poly(methylene-co-guanidine) polycations, protamine sulphate polycations, poly(allylamine) polycations (e.g., poly(allylamine hydrochloride) (PAH)), polydiallyldimethylammonium polycations, polyethyleneimine polycations, chitosan polycations, gelatine polycations, spermidine polycations and albumin polycations, poly[2-(dimethylamino)ethyl methacrylate], poly[N-3-methacrylamide], and poly[(dimethylamino)propyl acrylamide], among many others. The polycation preferably comprises one or more selected from the group consisting of poly(allylamine), polyethyleneimine, chitosan, gelatine, poly[2-(dimethylamino)ethyl methacrylate, poly[N-3-(dimethylamino)propyl methacrylamide], and poly[(climethylamino)propyl acrylamide].

The polycation can suitably have a number average molecular weight of at least 300 g/mol, such as in the range of 50 000-1 000 000 g/mol, in the range of 70 000-800 000 g/mol, in the range of 90 000-700 000 g/mol, or in the range of 100 000-500 000 g/mol.

Any anionic counter ions may be used in association with the polycation. Non-limiting examples of such counter ions include halides (such as chloride, fluoride, bromide, or iodide), sulphate, and methylsulphate.

Similar to the polycation, the polyanion can be a synthetic polymer or naturally occurring polymer. Examples of naturally occurring polyanions include glycosaminoglycans, such as condroitin sulphate, heparin, heparin sulphate, dermatan sulphate, keratin sulphate, and hyaluronic acid. The polyanion can also be a polysaccharide that can be chemically modified in order to incorporate a plurality of activated ester groups into the polysaccharide. Also acidic proteins having a net negative charge at neutral pH or proteins with a low isoelectric point can be used as naturally occurring polyanions. The anionic groups may be pendant to the polymer backbone, and/or may be incorporated into the polymer backbone. Optional thermoresponsive domains may be introduced into the polyanion by graft copolymerisation or block copolymerisation.

When the polyanion is a synthetic polymer, it is generally any polymer possessing anionic groups.

The polyanion can, for example, be a polyphosphate, such as a polyphosphate compound having 5-90 mol % of phosphate groups, such as 5-70 mol %, or 10-50 mol %. The polyphosphate can be a naturally occurring compound such as highly phosphorylated proteins like phosvitin (an egg protein), dentin (a natural tooth phosphoprotein), casein (a phosphorylated milk protein), or bone proteins (e.g. osteopontin).

The polyanion may also be a synthetic polypeptide. Such a synthetic polypeptide may, for example, be made by polymerising the amino acid serine and then chemically phosphorylating the polypeptide. One can also produce a polyphosphoserine by polymerisation of a phosphoserine. A polyphosphate can further be produced by chemically or enzymatically phosphorylating a protein (e.g., natural serine- or threonine-rich proteins), or by chemically phosphorylating a polyalcohol including, polysaccharides such as cellulose or dextran.

The polyphosphate can be a synthetic compound. For example the polyphosphate can be a polymer with pendant phosphate groups attached to the polymer backbone and/or present in the polymer backbone (e.g. a phosphodiester backbone).

The polyanion can also be a polyacrylate having one or more pendant phosphate groups. For example, the polyanion can be derived from the polymerisation of acrylate monomers including acrylates, methacrylates and the like. The polyanion may be a block-copolymer, where segments or portions of the copolymer possess anionic groups and neutral groups depending upon the selection of the monomers used to produce the copolymer.

The polyanion can include two or more sulphate, sulphonate, borate, boronate, carboxylate, phosphonate, or phosphate groups, combined with a plurality of activated ester groups.

Suitable examples of specific polyanions (excluding thermoresponsive moieties) include poly(styrene sulphonate) polyanions (e.g., poly(sodium styrene sulphonate) (PSS)), anionic polypeptides, poly(L-glutamic acid) polyanions, poly(D-glutamic acid) polyanions, anionic polysaccharides, dextran sulphate polyanions, heparin polyanions, polyacrylic acid polyanions, poly(2-acrylamido-2-methylpropane sulphonic acid), sodium alginate polyanions, Eudragit™ polyanions, gelatine polyanions, hyaluronic acid polyanions, carrageenan polyanions, xanthane polyanions chondroitin sulphate polyanions, cellulose sulphate polyanions, and carboxymethylcellulose polyanions, carboxymethyl starch polyanions among many others. The polyanion preferably comprises one or more selected from the group consisting of polyacrylic acid, poly(2-acrylamido-2-methylpropane sulphonic acid), sodium alginate, hyaluronic acid, carrageenan, chondroitin sulphate, and poly(styrene sulphonate).

The polyanion can suitably have a number average molecular weight of at least 300 g/mol, such as in the range of 50 000-1 000 000 g/mol, in the range of 70 000-800 000 g/mol, in the range of 90 000-700 000 g/mol, or in the range of 100 000-500 000 g/mol.

The amount of polycation in the adhesive complex coacervate composition of the invention can be 1-30% by total weight of the composition, such as 5-10%, or 10-15%. The amount of polyanion in the adhesive complex coacervate composition of the invention can be 1-30% by total weight of the composition, such as 5-10%, or 10-15%. Preferably, the mol ratio between polycation and polyanion is in the range of 0.8-1.2, such as in the range of 0.9-1.1.

The thermoresponsive moieties provide the polycation and/or the polyanion with a lower critical solution temperature (LCST). If the thermoresponsive moieties are only present in the polycations, then the polycations will be provided with a LCST. If the thermoresponsive moieties are only present in the polyanions, then the polyanions will be provided with a LCST. If the thermoresponsive moieties are present in both the polycations and the polyanions, then both will be provided with a LCST. The term “lower critical solution temperature” as used herein is meant to refer to moieties that are soluble in a liquid medium at a low temperature, but above a certain temperature (the LCST) precipitate from the liquid medium. At a temperature below the LCST, the polymer will display hydrophilic properties as a result of which the composition will be a liquid. At a temperature similar to or above the LCST, the polymer will display hydrophobic properties as a result of which the composition will phase separate.

Suitable examples of thermoreponsive moieties include poly(N-isopropylacrylamide), poly(N-isopropylacrylamide-co-allylamine), poly(N-isopropylacrylamide-co-trimethylaminoethylmethacrylate), poly(N-isopropylacrylamide-co-4-vinylbenzenesulphonate), polyetheramine, poly(2-isopropyl-2-oxazoline), poly (N,N-diethylacrylamide), poly(di(ethylene glycol) methacrylate), poly(N-vinylcaprolactam), poly[2-(climethylamino)ethyl methacrylate], poly(ethylene glycol), poly(N-n-propylacrylamide), poly(n-cyclopropylacrylamide), poly (N-(N′-isobutylcarbamido)propyl methacrylamide), poly(N-vinylisobutyramide), poly(N-vinyl-n-butyramide), poly(N-vinylpyrrolidone), polypropylene oxide), poly(oligo(ethylene glycol) methacrylate, poly[(di(ethylene glycol) ethyl ether acrylate)-co-(oligoethylene glycol acrylate)], oligo(ethylene oxide)-grafted polylactide, poly(methyl vinyl ether), poly(ethoxyethyl glycidal ether), poly(vinyl alcohol-co-vinyl acetal), poly(2-hydroxypropylacrylate), PEG-ylated poly-L-glutamate, P(Val-Pro-Gly-Val-Gly), poly(2-ethyl-2-oxazine), polylactic acid-co-ethylene glycol). It is also possible to use any combination of two or more of the above-mentioned thermoresponsive moieties. Preferably, the thermoresponsive moieties comprise one or more selected from the group consisting of poly(N-isopropylacrylamide), poly(ethylene glycol), polylactic acid-co-ethylene glycol), and poly(oligo(ethylene glycol) methacrylate).

Each thermoresponsive moiety in the polycation and/or the polyanion can have a number average molecular weight in the range of 500-100 000 g/mol, Preferably, each thermoresponsive moiety has a number average molecular weight in the range of 1000- 20 000 g/mol, more preferably in the range of 2000-10 000 g/mol.

In accordance with the invention, the polycation comprises 5-70 mol % of thermoresponsive moieties and/or the polyanion comprises 5-70 mol % of thermoresponsive moieties. The polycation in the adhesive complex coacervate composition of the invention may comprise 5-70 mol % of thermoresponsive moieties, preferably 10-60 mol %, such as 15-50 mol %. Similarly, the polyanion in the adhesive complex coacervate composition of the invention may comprise 5-70 mol % of thermoresponsive moieties, preferably 10-60 mol %, such as 15-50 mol %.

The lower critical solution temperature of the thermoresponsive moieties can be in the range of 0-70° C., preferably in the range of 10-60° C., more preferably in the range of 20-50° C., even more preferably in the range of 20-40° C. The exact LCST may be influenced or optimised, for instance, by the molar ratio of hydrophobic and hydrophilic fractions in the polymer, the molar mass of the polymer, the concentration of the polymer, and the pH and the ionic strength of the surrounding medium.

As an optional ingredient the adhesive complex coacervate composition of the invention may comprise one or more salts. These salts may be added to alter the ionic strength and thereby control the viscosity of the adhesive complex coacervate composition. Suitably, the concentration of the salt may be 0.01-3.0 M, such as 0.05-2.0 M, or 0.08-1.0 M, for example about 0.5 M, or about 0.1 M. Some examples of suitable salts that may be comprised in the adhesive complex coacervate composition include NaCl, MgCl₂, NaNO₃, K₂CO₃, NaI, and KI. The adjustable ionic strength thereby provides an additional delicate control over the physical state of the adhesive complex coacervate composition and the mechanical properties of its solid state.

The adhesive complex coacervate composition of the invention can comprise optional additives, such as one or more selected from the group consisting of silica nanoparticles, clay platelets, hairy nano- or microspheres, nanorods, magnetic nanop articles, catechol containing compounds, metal-legating copolymers, UV cross-linkable functional groups, and tackifiers. Preferably, the adhesive complex coacervate composition of the invention comprises one or more selected from the group consisting of silica nanoparticles, clay platelets, hairy nano- or microspheres, nanorods, and magnetic nanoparticles.

In a special embodiment, the adhesive complex coacervate composition of the invention further comprises one or more bioactive agents, preferably charged bioactive agents. Suitable bioactive agents may include one or more selected from the group consisting of drugs, amino acids, oligonucleotides, polypeptides (such as hormones, enzymes, and cytokines), genetic agents, proteins (growth factors), antigens, antibodies, vaccines, and anaesthetics.

In yet a further aspect the invention is directed to a method of solidifying an adhesive complex coacervate composition comprising heating an adhesive complex coacervate composition according to the invention above the lower critical solution temperature. Without wishing to be bound by any theory, the inventors believe that by heating the adhesive complex coacervate composition above the LCST the thermoresponsive domains self-aggregate which in turn leads to the formation of physical crosslinks in the material. This transition is reversible in the sense that upon cooling the material below the LCST the material becomes fluid again.

Heating may be done by any suitable heating means However, for in situ applications, body heat may suitably be used as a source for bringing the material above the LCST.

In yet a further aspect, the invention is directed to a physically crosslinked complex coacervate (or solid ionic gel) prepared from an adhesive complex coacervate composition according to the invention. Such preparation involves heating the adhesive complex coacervate composition above the LCST.

In yet a further aspect, the invention is directed to a method for adhering a tissue defect in a subject comprising contacting the tissue defect with the adhesive complex coacervate composition of the invention and heating the temperature of the adhesive complex coacervate composition above the lower critical solution temperature.

The method may preferably comprise administering the adhesive complex coacervate composition in liquid form to a subject, such as by using a needle. Such a needle can advantageously guide the adhesive composition to the location of interest, i.e. the tissue defect. For the purpose of administration, one or more salts may be added in order to increase the ionic strength and thereby decrease the viscosity of the adhesive complex coacervate. This may facilitate the administration. Preferably, the body temperature of the subject is used as heating source for heating the adhesive composition above the LCST. Hence, in a preferred embodiment the LCST of the polyanion and/or the polycation is below the body temperature of the subject. In case the subject is human, then the LCST of the polyanion and/or polycation is preferably less than 37° C. It is particularly preferred when the LCST of the polyanion and/or polycation is between room temperature and the body temperature of the subject, such as between 20° C. and 35° C. Optionally, further heating may be performed by external heating means.

Accordingly, in a further aspect, the invention is directed to the use of an adhesive complex coacervate composition as described herein as an in situ adhesive in a subject. This expression is meant to refer to the situation where the adhesive complex coacervate composition is used in situ (i.e. within the body) as adhesive for adhering material. In particular, such use may involve the repair of a tissue defect. Some examples of tissue include bodily vessel tissue, bladder tissue, bone tissue, brain tissue, breast tissue, bronchi tissue, diaphragm tissue, oesophagus tissue, gall bladder tissue, heart tissue, intestine tissue, kidney tissue, larynx tissue, liver tissue, lung tissue, lymph vessel tissue, lymph node tissue, nerve tissue, ovary tissue, pancreas tissue, prostate tissue, skin tissue, stomach tissue, and thyroid tissue, trachea tissue, urethra tissue, ureter tissue, uterus tissue, and vertebral disc tissue. In particular, the adhesive composition is suitable for repair of heart tissue, uterus tissue, subcutaneous tissue, brain tissue, lung tissue, breast tissue, kidney tissue, liver tissue, pancreas tissue, stomach tissue, and intestine tissue.

The invention has been described by reference to various embodiments, and methods. The skilled person understands that features of various embodiments and methods can be combined with each other.

All references cited herein are hereby completely incorporated by reference to the same extent as if each reference were individually and specifically indicated to be incorporated by reference and were set forth in its entirety herein.

The use of the terms “a” and “an” and “the” and similar referents in the context of describing the invention (especially in the context of the claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. For the purpose of the description and of the appended claims, except where otherwise indicated, all numbers expressing amounts, quantities, percentages, and so forth, are to be understood as being modified in all instances by the term “about”. Also, all ranges include any combination of the maximum and minimum points disclosed and include any intermediate ranges therein, which may or may not be specifically enumerated herein.

Preferred embodiments of this invention are described herein. Variation of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject-matter recited in the claims appended hereto as permitted by applicable law. Moreover, any combination of the above-described elements in all possible variations thereof is encompassed by the invention unless otherwise indicated herein or otherwise clearly contradicted by context. The claims are to be construed to include alternative embodiments to the extent permitted by the prior art.

For the purpose of clarity and a concise description features are described herein as part of the same or separate embodiments, however, it will be appreciated that the scope of the invention may include embodiments having combinations of all or some of the features described.

The invention will now be further illustrated by means of the following example, which is not intended to limit the scope in any manner.

### EXAMPLES

Poly(acrylic acid) (PAA, Mₙ=240 kDa), poly(N-isopropylacvrylamide) amine terminated (PNIPAM-NH₂, Mₙ=5.5 kDa), N,N′-dicyclohexylcarbodiimide (DCC), acrylic acid (AA), potassium persulphate (KPS) were purchased from Sigma. N,N-climethylaminopropyl acrylamide (DMAPPA, abcr GmbH) was passed through an alumina column to remove the inhibitor. Sodium metabisulphite (Na₂S₂O₅) was purchased from Scharlau All products except DMAPAA were used as received.

### Poly(acrylic acid)-grafted-poly(N-isopropylacryla,mide) synthesis

PAA-g-PNIPAM was synthesised as described by Durand (Polymer 1999, 40(17), 4941-4951) by coupling PAA and PNIPAM-NH₂ using DCC as a coupling agent. The copolymer was washed with methanol, purified by dialysis and recovered by freeze-drying. The mol % of PNIPAM sidechains was determined using ¹H-NMR. Copolymer Mₙ, was determined by size exclusion chromatography on Agilent Technologies 1200 system using a Ultrahydrogel 500 column with an Agilent 2100 RI detector. Samples were run in 100 mM NaNO₃, 10 mM phosphate, pH 7 and 0.5 ml/min flow rate. The number average Mₙ, was 400 kDa.

### Poly(N,N-dimethylaminopropyl acrylamide)-grafted-poly(N-isopropylacrylamide) synthesis

MacroPNIPAM was synthesised as described by Petit et al. (Polymer 2007, 48(24), 7098-7112) by coupling PNIPAM-NH₂ and AA using DCC as a coupling agent. PDMAPAA-g-PNIPAM was synthesised by free radical copolymerisation of DMAPAA and macroPNIPAM using the redox couple KPA and Na₂S₂O₅ as initiator. The copolymer was recovered by precipitation in acetonitrile, purified by dialysis and recovered by freeze drying. The mol % of PNIPAM sidechains was determined using ¹H-NMR. Poly(climethylaminopropyl acrylamide) (PDMAPAA) was synthesised using the same polymerisation technique. The procedure is identical, except for the absence of macroPNIPAM in the reaction mixture.

### Poly(N-isopropylacrylamide)-poly(acrylic acid)-poly(N-isopropylacrylamide block copolymer synthesis

2,2′-Azo-bis(2-methylpropionitrile (98%), N-isopropylacrylamide (97%), tert-butylacrylate (contains 10-20ppm monomethyl ether hydroquinone as inhibitor, 98%), trioxane 99.9%), concentrated hydrogen chloride (37% solution in water), and dioxane (anhydrous, 99.8%) were obtained from Sigma-Aldrich, Germany and used without further purification unless noted otherwise. N-isopropylacrylamide was recrystallised twice from a mixture of hexane and acetone. tert-Butylacrylate was passed over a short column of Al₂O₃ to remove the inhibitor. 2,2′-Azo-bis(2-methylpropionitrile was recrystallised thrice from methanol. Bis(2-methylpropionic acid)trithiocarbonate and poly(N-isopropylacrylamidebis(2-methylpropionic acid)trithiocarbonate were synthesised as described by Lai et al. (Macromolecules 2002, 35(18), 6754-6756). Hexafluoroisopropanol and methanol were obtained from Biosolve, France.

A Schlenk flask was charged with a solution of poly(N-isopropylacrylamide₂₄-bis(2-methylpropionic acid)trithiocarbonate (0.581 g, 0.104 mmol), tert-butylacrylate (5.3 g, 41.4 mmol), 2,2′-azo-bis(2-methylpropionitrile (3.4 mg, 0.020 mmol) and trioxane (0.372 g, 4.14 mmol) in dioxane (21 ml), and was subjected to five freeze-pump-thaw cycles. The solution was magnetically stirred at 70° C. for 45 min, after which the polymerisation was stopped by admitting atmosphere into the flask while cooling on an ice bath. The material precipitated thrice in ice-cold methanol:water 3:1 and dried under high vacuum.

Poly(N-isopropylacrylamide)-b-poly(tert-butylacrylate)-bis(2-methylpropionic acid)trithiocarbonate (3.5 g, 0.093 mmol) was dissolved in hexafluoroisopropanol (HFIP) (314 ml) in a round-bottomed flask, to which concentrated hydrogen chloride (HCl) 37% (2.6 ml, 31.4 mmol) was added, 1.3 equivalents with respect to the amount of tert-butyl units. After 4 h, the mixture was stripped of volatiles and the dry polymer was dispersed in water and titrated with a sodium hydroxide solution until the solution was neutral. A slight cloudiness was removed by centrifugation at 15 000×g, and the supernatant was dehydrated by freeze drying. Poly(acrylic acid)-b-poly(N-isopropylacrylamide)-bis(2-methylpropionic acid)trithiocarbonate (corresponding to poly(N-isopropylacrylamide)-poly(acrylic acid)-poly(N-isopropylacrylamide)) was recovered as a white fluffy powder.

### Poly(N-isopropylacrylamide)-grafted-poly(N-[3-(dimethylamino)propyl] methacrylamide copolymer synthesis

Amino terminated poly(N-[3-(dimethylamino)propyl] methacrylamide) (PMADAP-NH₂) was prepared in cold water using potassium persulphate (KPS) as the initiator and 2-aminoethanethiol hydrochloride (AET-HCl) as the chain transfer agent. The macromonomer was synthesised through a coupling reaction with acrylic acid (AA) in N-methyl-2-pyrrolidone (NMP), where double bonds were introduced onto the chain ends. In a grafting through approach, radical polymerisation of MADAP macromonomer and N-isopropylacrylamide (NIPAM) was performed in NMP using azobisisobutyronitrile (AIBN) at 70° C. At the end of the reaction water was then added in a ratio of 2:1 compared to NMP and dialysis was performed for a week. The polymer wad then recovered by freeze drying.

The syntheses was followed by ¹H-NMR in deuterium oxide (D₂O), which determined the ratio of the ionisable comonomers (side chains) in PNIPAM-g-PMADAP to be around 32 wt.% (corresponding to 24 mol %).

### Poly(N-isopropylacrylamide)-grafted-poly(2-acrylamido-2-methylpropane sulphonic acid) copolymer synthesis

Amino terminated poly(2-acrylamido-2-methylpropane sulphonic acid) (PAMPS-NH₂) was prepared in cold water using KPS as the initiator and AET-HCl as the chain transfer agent. Since the preparation of the macromonomer proved infeasible, a grafting onto approach was followed. At first, a random copolymer of P(NIPAM-AA) containing a small amount of acrylic acid (5%) was synthesised in cold water using KPS as the initiator and AET-HCl as the chain transfer agent. Subsequently, A coupling reaction between the amino-terminated PAMPS telomers and the AA units along the backbone was performed in water using 1-ethyl-3-(3-climethylaminopropyl)carbodiimide/N-hydroxysuccinimide (EDC/NHS) coupling. Dialysis was then performed for one week and the PNIPAM-g-PAMPS graft copolymer was recovered by freeze drying

The synthesis was followed by ¹H-NMR in deuterium oxide (D₂O), which determined the ratio of the ionizable comonomers (side chains) in PNIPAM-g-PAMPS to be around 64 wt.% (corresponding to 49 mol %).

### Coacervate Formation

Stock solutions of PAA-g-PNIPAM and PDMAPAA-g-PNIPAM were prepared at a chargeable monomer concentration of 0.1 M. The pH of PAA-g-PNIPAM solution was adjusted to 7.0 using 0.1 M NaOH and 0.1 M HCl. Afterwards, a calculated amount of PDMAPAA-g-PNIPAM solution was mixed with a calculated amount of 3.0 M NaCl and milli-Q water in a centrifuge tube. The pH of the mixture was adjusted to 7.0. Finally, a calculated amount of PAA-g-PNIPAM solution was added to the mixture to reach a 0.05 M total charged monomer concentration, a 0.5 mixing ratio and a 0.75 M NaCl concentration. Complex coacervation took place after addition of PAA-g-PNIPAM solution. After vigorous shaking, the coacervate phase was homogeneously dispersed throughout the mixture. The mixture was left to equilibrate for 1 day and then it was centrifuged at 4000×g for 1 hour. Two clearly separated phases appeared, with the coacervate phase precipitated at the bottom of the centrifuge tube. In addition to that, complex coacervates were prepared mixing homopolymer solutions (PAA and PDMAPAA). These samples were obtained using the same procedure and the same parameters described above.

### Water Content

The water content was determined by freeze-drying the coacervate phase and weighing the recovered amount on a Mettler Toledo XC205DY analytical balance. The water content was determined by the weight difference before and after the freeze-drying process. All measurements were done by triplicate to ensure data reproducibility.

### Dynamic Rheology

The storage (G′) and the loss moduli (G″) were measured as a function of angular frequency and temperature with a cone and plate configuration (25 mm diameter, 1° angel cone) on a stress-controlled rheometer (Anton Paar MCR301). Frequency sweeps were performed at 20° C. and at 50° C. at a constant strain of 0.5% in a frequency range between 0.1 and 100 rad/s. Temperature sweeps were performed at a frequency of 1 rad/s and at a strain of 0.5% as the temperature was increased from 0° C. to 70° C. at a rate of 1° C/min. All measurements were done by triplicate to ensure data reproducibility.

Underwater adhesion test

Underwater adhesion properties were measured using a tack test setup developed by Sudre et al. (Soft Matter 2012, 8(31), 8184-8193) and mounted on a Instrom 5565 materials testing system with a 10 N load cell. The test consists on making a parallel contact and detachment underwater between a homogeneous layer of the coacervate (thickness˜2 mm) and a PAA thin film (thickness˜200 nm). PAA hydrogel thin films were synthesised by simultaneously crosslinking and grafting reactive PAA by thiol-ene reaction, according to the protocol described by Chollet et al. (ACS Applied Materials 2016, 8(18), 11729-11738). The thin film was attached on a mobile stainless steel probe, which was fixed to the load cell. The coacervate sample was glued with a cyanoacrylate adhesive (LOCTITE™ 425) onto a glass slide inside a polydimethylsiloxane (PDMS) mould (thickness˜2.5 mm). Subsequently, water was poured in the chamber, with the salt concentration and the pH of medium matching the ones of the sample (pH 7.0, 0.75 M NaCl). Contact between the clean PAA thin film and the coacervate was performed underwater at 20° C. The setup was covered at the top with a rubber layer that could provide heat insulation and temperature control. Detachment was then performed either at 20° C. or at 50° C. after a contact time of 900 s at a constant debonding speed of 50 μm/s. Raw data of force and displacement were converted into stress and strain values to obtain the work of adhesion. The strain ϵ was obtained by normalising the displacement by the initial thickness of the sample (T₀˜2 mm). The normalised stress σ was obtained by dividing the force by the thin film contact area. The work of adhesion W_{adh} was then calculated as follows.

W_{adh}=T₀∫₀^{ϵ}^{ₘₐₓ}σdϵ

All measurements were done by duplicate to ensure data reproducibility.

### Results

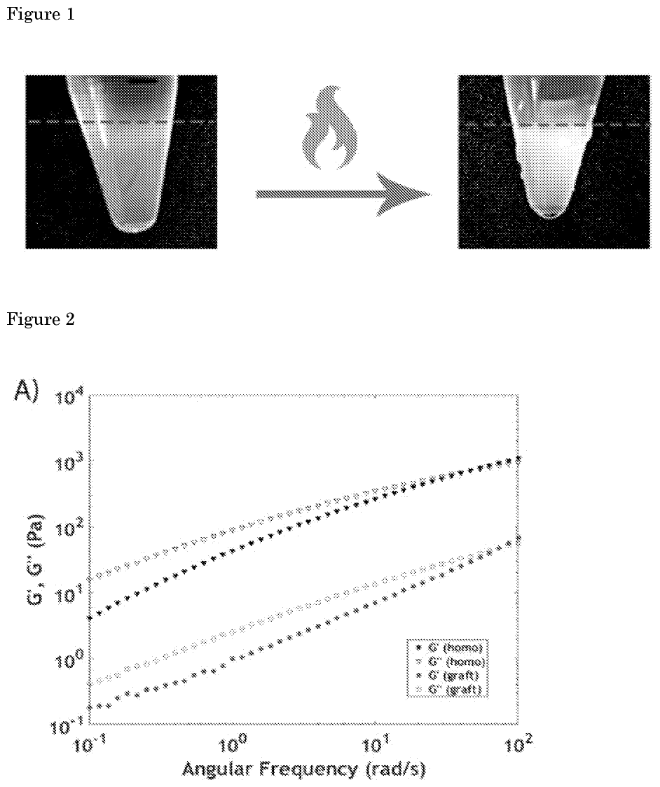

Temperature induced complex coacervation of an adhesive complex coacervate composition comprising PAA-g-PNIPAM and PDMAPAA-g-PNIPAM is shown in FIG. 1. Upon heating the liquid adhesive complex coacervate composition above the LCST, the composition self-assembles into a tough solid.

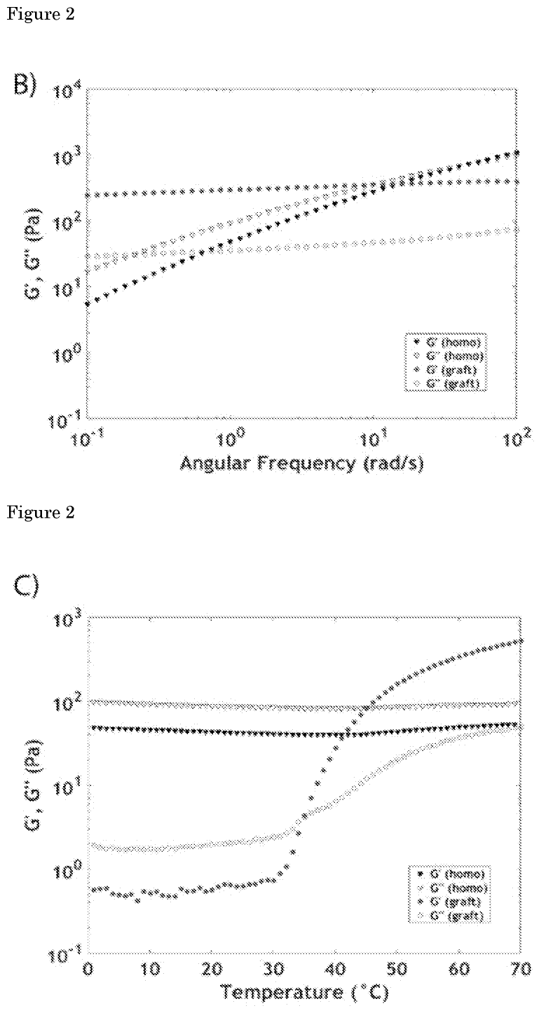

Rheological measurements were performed on the adhesive complex coacervate composition as a function of frequency and temperature. At 20° C. (FIG. 2A), both compositions prepared from homopolymer and graft copolymer solutions possess a fluid character with the loss modulus (G″) overcoming the storage modulus (G′) up to high frequencies, where the crossover is visible. However, in graft copolymers compositions the crossover frequency is higher (˜70 rad/s) when compared to homopolymer compositions (˜45 rad/s), while both moduli are lower in the whole frequency range this means that PNIPAM side chains act as a sort of plasticiser between the polyelectrolyte backbones, allowing faster relaxation times and weakening strength of the electrostatic interactions. At 50° C. (FIG. 2B), no differences can be observed in the coacervates prepared from homopolymer solutions, meaning that temperature does not significantly affect the viscoelastic properties of the material. However, the samples prepared from graft copolymer solutions, initially transparent and fluid-like, turned white and solid-like when the temperature was raised above 30° C., the LCST (FIG. 1). The rheological data obtained at 40° C. show that both moduli increase and become frequency independent, with G′ overcoming G″ (FIG. 2B). This indicates that the adhesive complex coacervate composition, upon the increase in temperature, behaves as a highly interconnected gel network. Without wishing to be bound by any theory, the inventors believe that this transition can be attributed to the presence of PNIPAM domains, which self-aggregate when the temperature is raised above the LCST, leading to the formation of physical crosslinks in the material. The transition is reversible since the coacervate recovers the original. morphology when cooled below the LCST, such as to 4° C.

In order to accurately detect the transition, temperature sweeps were performed on the samples, heating the sample from 0° C. to 70° C. (FIG. 2C).

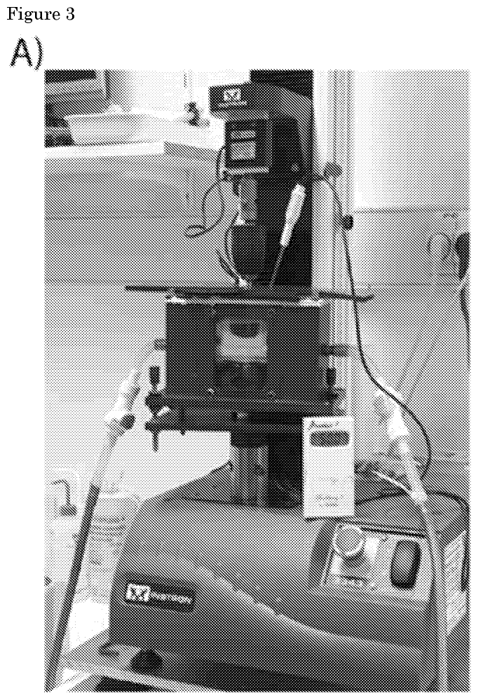

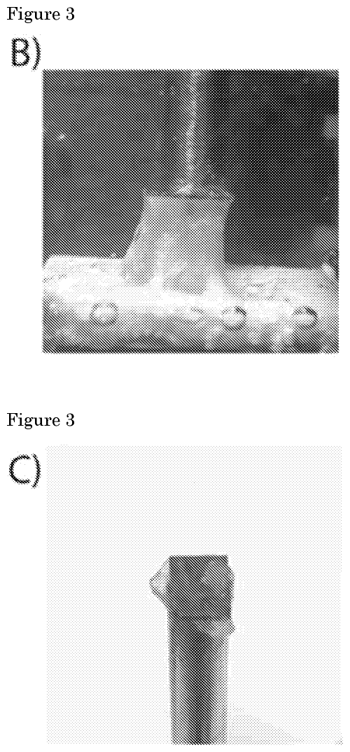

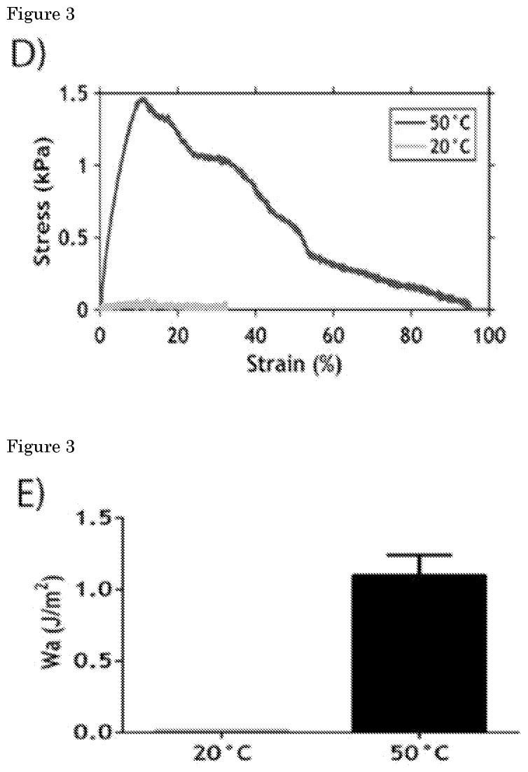

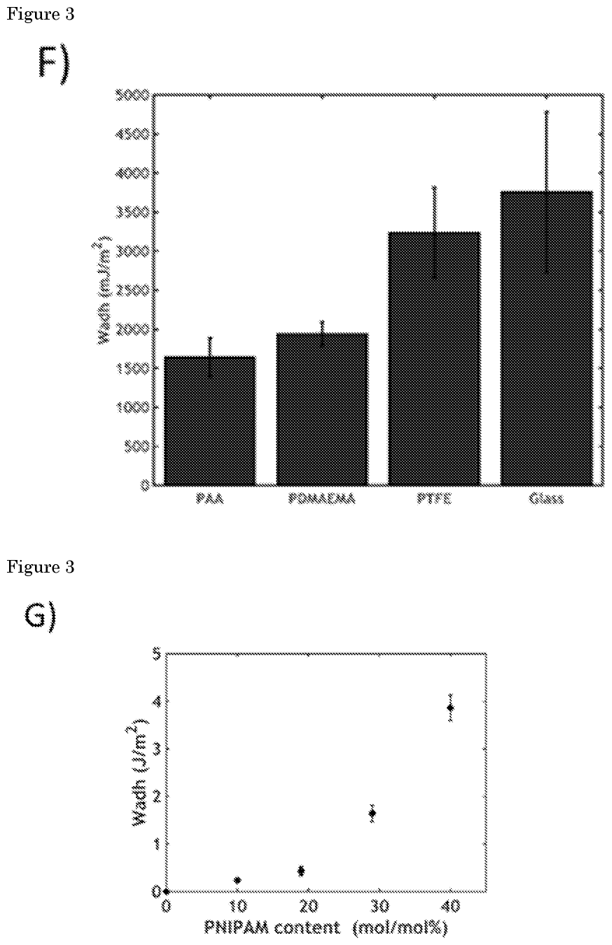

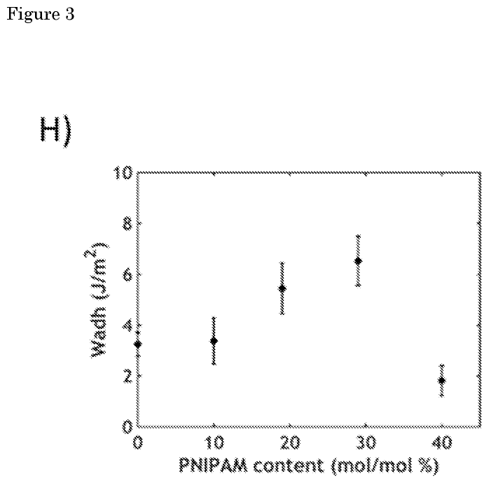

Underwater adhesion experiments were conducted on coacervates prepared both from homopolymer and graft copolymer solutions using a methodology not yet employed by underwater adhesives developers. A probe-tack test was performed completely underwater using the setup developed by Sudre et al. (Soft Matter 2012, 8(31), 8184-8193) (FIG. 3A). Contact with a PAA hydrogel thin film was made underwater at 20° C. while detachment was performed either at 20° C. or at 50° C. Coacervates prepared from homopolymer solutions can reach pretty high strain values but cannot sustain any stress: no resisting force could be detected by the load cell upon detachment, meaning that the work of adhesion (W_{adh}) is close to zero. The samples are viscous enough to provide good contact with the probe, but they cannot bear any load because no curing process has been introduced. In addition to that, no difference in adhesion can be observed upon raising the temperature to 50° C. since no thermoresponsive moiety is present in the material.

A similar trend is observed when probing the underwater adhesion properties of coacervates prepared from graft copolymer solutions at 20° C.: forces close to the load cell sensibility are detected, leading to a pretty low value of W_{adh} (0.02 J/m²). However, when detachment is performed at 50° C., the formation of new physical crosslinks provide additional strength to coacervate (FIG. 3B), resulting in an increase of two orders of magnitude in W_{adh} (1.10 J/m²) (FIG. 3E). The stress-strain curve recorded consists of a stress peak, followed by a continuous decrease in stress until complete detachment of the probe (FIG. 3D). The mode of failure is prevalently cohesive (FIG. 3C): at the end of the test, residues of the material can be found on the probe. It follows that stronger interactions could be introduced in the material to improve its cohesion even further.

Another parameter that plays a key role in the adhesion performance is the interaction between the sample and the probe surface. The complex coacervate adheres strongly to both hydrophilic (glass) and hydrophobic surfaces (polytetrafluoroethylene, PTFE), providing higher W_{adh} (work of adhesion) values than using the negatively charged PAA surface (FIG. 3F).

The same probe-tack experiments were performed by using, as a probe, a positively charged brush, obtained by attaching poly(climethylaminoethyl methacrylate) (PDMAEMA) chains to the probe surface, and a similar work of adhesion and probe-tack curve as with the negatively charged PAA surface were obtained (FIG. 3F).

The composition of the graft copolymers was altered to study the effect of composition on the work of adhesion (FIG. 3G). By increasing the amount of PNIPAM in the graft copolymers relative to the amount of polyion, while keeping the graft length constant, a significant increase in the work of adhesion was obtained at an ionic strength of 0.75 M.

Similar experiments were performed at a lower salt concentration, 0.1 M NaCl. Again the composition of the graft copolymers was altered to study the effect of composition on the work of adhesion (FIG. 3H). By increasing the amount of PNIPAM in the graft copolymers to 30% relative to the amount of polyion, while keeping the graft length constant, a significant increase in the work of adhesion was obtained. A further increase to 40% resulted in a decrease in work of adhesion.

1. An adhesive complex coacervate composition comprising a polycation and a polyanion, wherein said polycation and polyanion together comprise on average at least two thermoresponsive moieties per polymer chain, said thermoresponsive moieties exhibiting a lower critical solution temperature, wherein said polycation comprises 5-70 mol % of thermoresponsive moieties and/or wherein said polyanion comprises 5-70 mol % of thermoresponsive moieties, and wherein said polycation and/or said polyanion is a graft copolymer or block copolymer comprising said thermoresponsive moieties.

2. The adhesive complex coacervate composition according to claim 1, wherein said polycation comprises 5-70 mol % of thermoresponsive moieties and said polyanion comprises 5-70 mol % of thermoresponsive moieties.

3. The adhesive complex coacervate composition according to claim 1, wherein said polycation is a graft copolymer or block copolymer comprising thermoresponsive moieties.

4. The adhesive complex coacervate composition of claim 1, wherein said polyanion is a graft copolymer or block copolymer comprising thermoresponsive moieties.

5. The adhesive complex coacervate composition of claim 1, wherein said polycation comprises at least two thermoresponsive moieties and said polyanion comprises at least two thermoresponsive moieties.

6. The adhesive complex coacervate composition of claim 1, wherein said polycation comprises one or more selected from the group consisting of poly(allylamine), polyethyleneimine, chitosan, gelatine, poly[2-(dimethylamino)ethyl methacrylate], poly[N-3-(dimethylamino)propyl methacrylamide] and poly[(dimethylamino)propyl acrylamide].

7. The adhesive complex coacervate composition of claim 1, wherein said polycation has a number average molecular weight in the range of 50 000-1 000 000 g/mol.

8. The adhesive complex coacervate composition of claim 1, wherein said polyanion comprises one or more selected from the group consisting of polyacrylic acid, poly(2-acrylamido-2-methylpropane sulfonic acid), sodium alginate, hyaluronic acid, carrageenan, chondroitin sulphate, and poly(styrenesulphonate).

9. The adhesive complex coacervate composition of claim 1, wherein said polyanion has a number average molecular weight in the range of from 50 000-1 000 000 g/mol.

10. The adhesive complex coacervate composition of claim 1, wherein said thermoresponsive moieties comprises one or more selected from the group consisting of poly(N-isopropylacrylamide), poly(N-isopropylacryla mide-co-allylamine), poly(N-isopropylacrylamide-co-trimethylaminoethylmethacrylate), poly(N-isopropylacrylamide-co-4-vinylbenzenesulphonate), polyethera mine, poly(2-isopropyl-2-oxazoline), poly(N,N-diethylacrylamide), poly(di(ethylene glycol)methacrylate), poly(N-vinylcaprolactam), poly[2-(dimethylamino)ethyl methacrylate], poly(ethylene glycol), poly(N-n-propylacrylamide), poly(n-cyclopropylacrylamide), poly(N-(N′-isobutylcarbamido)propyl methacrylamide), poly(N-vinylisobutyramide), poly(N-vinyl-n-butyramide), poly(N-vinylpyrrolidone), poly(propylene oxide), poly(oligo(ethylene glycol) methacrylate, poly[(di(ethylene glycol) ethyl ether acrylate)-co-(oligoethylene glycol acrylate)], oligo(ethylene oxide)-grafted polylactide, poly(methyl vinyl ether), poly(ethoxyethyl glycidal ether), poly(vinyl alcohol-co-vinyl acetal), poly(2-hydroxypropylacrylate), PEG-ylated poly-L-glutamate, P(Val-Pro-Gly-Val-Gly), poly(2-ethyl-2-oxazine), and poly(lactic acid-co-ethylene glycol.

11. The adhesive complex coacervate composition of claim 1, wherein said polycation comprises 10-60 mol % of thermoresponsive moieties.

12. The adhesive complex coacervate composition of claim 1, wherein said polyanion comprises 10-60 mol % of thermoresponsive moieties.

13. The adhesive complex coacervate composition of claim 1, wherein said lower critical solution temperature is in the range of 0-70° C.

14. The adhesive complex coacervate composition of claim 1, wherein the adhesive complex coacervate composition further comprises one or more selected from the group consisting of silica nanoparticles, clay platelets, hairy nano- or micro spheres, nanorods, magnetic nanoparticles, catechol containing compounds, metal-legating copolymers, UV cross-linkable functional groups, and tackifiers.

15. The adhesive complex coacervate composition of claim 1, further comprising one or more bioactive agents.

16. A method of physically crosslinking an adhesive complex coacervate composition comprising heating the adhesive complex coacervate composition of claim 1 above the lower critical solution temperature.

17. A method for adhering a tissue defect in a subject comprising contacting the tissue defect with the adhesive complex coacervate composition of claim 1 and heating the temperature of the adhesive composition above the lower critical solution temperature.

18. (canceled)

19. The adhesive complex coacervate composition of claim 1, wherein the thermoresponsive moieties comprise one or more selected from the group consisting of poly(N-isopropylacrylamide), poly(ethylene glycol), poly(lactic acid-co-ethylene glycol), and poly(oligo(ethylene glycol) methacrylate.

20. The adhesive complex coacervate composition of claim 15, wherein said one or more bioactive agents are selected from the group consisting of drugs, amino acids, oligonucleotides, polypeptides, hormones, enzymes, cytokines, genetic agents, proteins, growth factors, antigens, antibodies, vaccines, and anaesthetics.
